(19) United States
(12) Patent Application Publication
(10) Pub. No.: US 2008/0085333 A1
(43) Pub. Date: Apr. 10, 2008

(54) Composition Comprising Extract of Anemarrhena Asphodeloides and Aralia Elata,and Use Thereof

(75) Inventors: Eun-Kyung Hong, Seoul (KR); Young-Shin Chung, Seoul (KR)

Correspondence Address: BUCHANAN, INGERSOLL & ROONEY PC, POST OFFICE BOX 1404, ALEXANDRIA, VA, 22313-1404, US

(73) Assignee: MEDVILL CO., LTD., Seoul (KR)

(21) Appl. No.: 11/665,704

(22) PCT Filed: Oct. 18, 2005
(86) PCT No.: PCT/KR05/03466

§ 371 Date: Apr. 18, 2007

(30) Foreign Application Priority Data
Oct. 19, 2004 (KR) 10-2004-0083709

(51) Int. Cl. A61K 36/00; A61K 36/25; A61P 17/00

(52) U.S. Cl. 424/773; 424/725

(57) ABSTRACT

The present invention relates to a composition comprising a water or organic solvent extract of Anemarrhena asphodeloides and Aralia elata, and the use thereof. The composition has a preventive or therapeutic effect on inflammatory skin diseases and an antibacterial effect against Propionibacterium acnes.

### TECHNICAL FIELD

This application claims priority to Korean Patent Application No. 10-2004-83709, filed on Oct. 19, 2004, the contents of which are hereby incorporated by reference.

The present invention relates to a composition comprising a water or organic solvent extract of Anemarrhena asphodeloides and Aralia elata, and use thereof. More particularly, the present invention relates to a composition comprising a water or organic solvent extract of Anemarrhena asphodeloides and Aralia elata, the use thereof for preventing or treating inflammatory skin diseases, and the antibacterial use thereof against Propionibacterium acnes.

### BACKGROUND ART

Inflammatory skin diseases are referred to diseases accompanied with a series of clinical signs and symptoms, such as itch, edema, erythema and abrasion are induced by various stimulative factors that cause a series of inflammatory reactions in the skin epithelium.

As the inflammatory skin diseases, atopic dermatitis, contact dermatitis, seborrheic dermatitis, acne, etc., are known. The atopic dermatitis is generally used in the same meaning as eczema and is an eczema-like skin lesion occurring in persons having atopic constitution. It is also called endogenous eczema or Besnier's prurigo. The cause of the atopic dermatitis is not yet found but is known to involve genetic factors, and at the present time, the dominant view is that the atopic dermatitis is a kind of autoimmune disease. Unlike common eczema or dermatitis, the atopic dermatitis shows specific symptoms and progression, accounts for 70-80% of childhood eczema and recently, often occurs in adults as well.

The contact dermatitis' is a skin inflammation, which occurs when foreign substances are in contact with the skin. Although it shows symptoms like acute eczema, it is different from eczema, in that it occurs by a response to a certain foreign substance.

The seborrheic dermatitis is a dermatitis that frequently occurs on areas with a high sebum secretion, such as the scalp, the forehead and the armpit, and is also called seborrheic eczema. It causes much erythema and fine scale (dandruff) and often appears in persons in the 20-40 age group. Unlike common eczema, it is a disease resulting from abnormal constitution or sebum secretion, and is characterized in that it causes the skin to be sensitive to sunlight or heat, grows worse mainly in spring and autumn and tends to recur.

Acne is a chronic inflammatory disease occurring in hair follicles and sebaceous glands, and is considered to occur mainly by an increase in sebum secretion and the proliferation of Propionibacterium acnes, anaerobic skin flora. Also, it is sometimes caused by the complex action of various mechanisms. Sebum in a region where acne often occurs is produced by a mechanism where testosterone, a male sex hormone, is converted into dihydrotestosterone, an active form, by 5α-reductase, and sebum is excessively secreted by the action of the hormone. The excessive sebum produced is accumulated in hair follicles to clog the hair follicles so that the sebum is converted into free fatty acids and various low-molecular-weight substances by lipase and chemofactic factors produced by Propionibacterium acnes, anaerobic skin flora. Thereby gathering leucocytes around the hair follicles, and they destroy the hair follicle wall, the follicle contents will flow out into the dermis, thus causing an inflammatory reaction.

Until now, antihistamine agents, vitamin ointments and adrenal hormone preparations are frequently used for treating the inflammatory skin diseases. However, these drugs mostly have temporary effects and often show severe side effects.

Particularly in the case of the atopic dermatitis, a variety of 5-lipoxygenase inhibitors have been suggested as candidate compounds for antiallergic agents, and cromolyn is known to make the reaction between allergens and tissue mast cells ineffective so as to relief symptoms. However, these substances have a problem in that their clinical effects are unclear.

For the treatment of acne, methods of either using antibiotic agents, such as erythromycin, or controlling sebum by the use of estrogen, a female sex hormone, have been used, but these have a problem in that they side effects. In cosmetics for the treatment of acne, vitamin A derivatives, benzoyl peroxide, salicylic acid, triclosan and the like have been used and show some antibacterial effects, but these substances have the problem of causing side effects, including skin redness, skin hypersensitivity or light hypersensitivity.

### DETAILED DESCRIPTION OF THE INVENTION

Technical Problem

Accordingly, the present inventors have conducted many studies to develop a side-effect-free composition capable of effectively preventing or treating inflammatory skin diseases and as a result, found that a water or organic solvent extract of Anemarrhena asphodeloides and Aralia elata is significantly effective in preventing or treating inflammatory skin diseases, as compared to a single extract, and shows no toxicity and thus can be safely used in vivo. On the basis of this finding, the present invention has been completed.

Therefore, it is an object of the present invention is to provide a composition comprising a water or organic solvent extract of Anemarrhena asphodeloides and Aralia elata and the use thereof.

Technical Solution

To achieve the above object, in one aspect, the present invention provides a composition comprising a water or organic solvent extract of Anemarrhena asphodeloides and Aralia elata.

In another aspect, the present invention provides a pharmaceutical composition for preventing or treating inflammatory skin diseases, which comprises a composition comprising a water or organic solvent extract of Anemarrhena asphodeloides and Aralia elata.

In still another aspect, the present invention provides an antibacterial composition against Propionibacterium acnes, which comprises a composition comprising a water or organic solvent, extract of Anemarrhena asphodeloides and Aralia elata.

In still another aspect, the present invention provides a method for preventing or treating inflammatory skin diseases, which comprises administering to a subject in need thereof an effective amount of a composition comprising a water or organic solvent extract of Anemarrhena asphodeloides and Aralia elata.

In still another aspect, the present invention provides a method for inhibiting the growth of Propionibacterium acnes, which comprises administering to a subject in need thereof an effective amount of a composition comprising a water or organic solvent extract of Anemarrhena asphodeloides and Aralia elata.

In still another aspect, the present invention provides a water or organic solvent extract of Anemarrhena asphodeloides and Aralia elata for use as an active therapeutic ingredient.

In still another aspect, the present invention provides the use of a composition comprising a water or organic solvent extract of Anemarrhena asphodeloides and Aralia elata) for preparing an agent preventing or treating inflammatory skin diseases.

In yet another aspect, the present invention provides the use of a composition comprising a water or organic solvent extract of Anemarrhena asphodeloides and Aralia elata, for preparing an antibacterial agent against Propionibacterium acnes.

Hereinafter, the present invention will be described in detail.

Unless otherwise defined, all the technical and scientific terms used herein have the same meanings as commonly understood by those ordinary skill in the art to the present invention pertains.

The composition according to the present invention is characterized by comprising a water or organic solvent extract of Anemarrhena asphodeloides and Aralia elata, as an active ingredient. Because the inventive composition contains the water or organic solvent extract of Anemarrhena asphodeloides and Aralia elata, it has a synergistic effect on the prevention or treatment of inflammatory skin diseases as compared to a single extract of each plant.

As used herein, the term “synergistic effect” means that the effect arising in the combined use of extracts is higher than the sum of the effects occurring in the single use of each extract.

Anemarrhena asphodeloides is a perennial plant belonging to the Liliaceae family and is native to China. In Korea, it is cultivated in the central area of the country. Generally, its dried rhizome has been used as an herbal medicine and is known to have anti-inflammatory, fever-alleviating, antidiarrheal, diuretics effect, lumbago alleviation and suppression effects. Anemarrhena asphodeloides is known to contain active ingredients, including 6% asphonin, steroid sapogenins, such as sarsasaponin and markogenin (2-hydroxy sarsasapogenin), flavonoids and tanin. It is disclosed in Korean patent publication No. 2001-76516 that an extract of Anemarrhena asphodeloides has an excellent antibacterial effect against Propionibacterium acnes and thus can be used for the prevention or treatment of acne.

Aralia elata is a plant belonging to the Araliaceae family and is a perennial plant growing naturally in East Asia. In Chinese medicine, the root, fruit and bark of Aralia elata have been used for diabetes, kidney disease, acute hepatitis, rheumatoid arthritis, stomach cancer and gastrointestinal disorders. Particularly, in oriental medicine handbook (DongEuiBoGam; edited by Hur-Jun in Korea in the year of 1613), the dried root of bark of Aralia elata were used for diabetes, a headache, colic, colitis and gastric ulcer and as a tonic. In folk remedies, the Whole plant of Aralia elata has been used for gastrointestinal disorders. The bark of Aralia elata contains various triterpenoids, including saponin, and the cortex of Aralia elata contains a number of glycosides, including elatoside E having hypoglycaemic effects, elatoside F and olenolic acid glycosides, and also elatosides A and B that inhibit ethanol absorption (Yoshikawa M. et al., Chem. Pharm. Bull., 41:2069-2071, 1993).

To examine the anti-inflammatory effect of the water or organic solvent extract of Anemarrhena asphodeloides and Aralia elata, the present inventors measured the inhibitory effect of the extract on carrageenin-induced mouse paw edema and the inhibitory effect of the extract on PGE2 production in mouse macrophages (see Test Example 1). From the test results, it could be seen that the water or organic solvent extract of Anemarrhena asphodeloides and Aralia elata, according to the present invention, effectively inhibited carrageenin-induced mouse paw edema as compared to a single extract of each plant (see Table 1), and had an excellent effect on the inhibition of PGE2 production in mouse macrophages (see Table 2). Also, the effect of the water or organic solvent extract of Anemarrhena asphodeloides and Aralia elata was shown to be higher than the sum of the effects occurring when an extract of Anemarrhena asphodeloides and an extract of Aralia elata were administered alone. This indicates that the inventive extract has a synergistic effect.

Furthermore, the present inventors examined if the inventive water or organic solvent extract of Anemarrhena asphodeloides and Aralia elata has an antibacterial effect against Propionibacterium acnes (see Test Example 3). As a result, it could be found that the inventive extract had a very excellent antibacterial effect against Propionibacterium acnes, in that it was 6 to 20-fold higher in inhibitory activity against Propionibacterium acnes, than that of a single extract of Anemarrhena asphodeloides or Aralia elata (see Table 4).

Anemarrhena asphodeloides and Aralia elata contained in the inventive extract are collected from nature or commercially available. Anemarrhena asphodeloides and Aralia elata used in the present invention may be the whole plant parts, and preferably rhizomes in the case of Anemarrhena asphodeloides, and stems in the case of Aralia elata.

Anemarrhena asphodeloides and Aralia elata used for the preparation of the inventive extract are preferably used dried body thereof, and may be used after pulverization in order to increase extraction efficiency. As methods of drying Anemarrhena asphodeloides and Aralia elata, drying in the sun, drying in the shade, hot-air drying, freeze drying and natural drying may all be used. Preferably, hot-air drying and freeze drying may be used.

The inventive water or organic solvent extract of Anemarrhena asphodeloides and Aralia elata can be prepared by either extracting Anemarrhena asphodeloides and Aralia elata together or extracting each of Anemarrhena asphodeloides and Aralia elata depending on the physical and chemical properties of the pharmacologically effective ingredients thereof and then mixing the extracts with each other. Preferably, the inventive extract can be prepared by either pulverizing the dried Anemarrhena asphodeloides and Aralia elata together to prepare powder and then extracting the powder, or mixing Anemarrhena asphodeloides and Aralia elata powders with each other at a predetermined ratio and then extracting the powder mixture. In this regard, the dry weight ratio of Anemarrhena asphodeloides and Aralia elata is preferably 1-10:1-15, more preferably 1-5:1-10, and most preferably 3:2.

In one test example of the present invention, an inhibitory effect on carrageenin-induced mouse paw edema according to the weight ratio of Anemarrhena asphodeloides and Aralia elata was examined (see Test Example 2). As a result, it was shown that extracts prepared using dried Anemarrhena asphodeloides powder and dried Aralia elata powder at dry weight ratios of 1-5:1-10 all had an excellent effect on the inhibition of mouse paw edema. Particularly, the use of an extract prepared using the dried Anemarrhena asphodeloides powder and the dried Aralia elata powder at a dry weight ratio of 3:2 showed the highest inhibitory effect on mouse paw edema (see Table 3).

The preparation of the water or organic solvent extract of Anemarrhena asphodeloides and Aralia elata may be performed by any method known in the art. In other words, the inventive extract may be prepared by cutting the plants into a given size, and then either extracting the cut material with an extraction solvent, followed by filtration, concentration and drying, or heating the cut material in an extraction solvent for at least two hours, followed by filtration and concentration.

Examples of the extraction solvent used may various solvents include water and alcohols, such as ethanol and methanol. Preferably, water or ethanol may be used in the preparation of the inventive extract.

In one examples of the present invention, a water extract of Anemarrhena asphodeloides and Aralia elata (see Example 2) and an ethanol extract of Anemarrhena asphodeloides and Aralia elata (see Example 3) were prepared. These extracts were compared to each other for their anti-inflammatory effects and as a result, it was shown that the water extract of Anemarrhena asphodeloides and Aralia elata was slightly higher in the anti-inflammatory effect than that of the ethanol extract but had no significant difference (see Test Example 1). This suggests that the inventive water or organic solvent extract of Anemarrhena asphodeloides and Aralia elata shows anti-inflammatory activity regardless of the extraction solvent.

Most preferably, the inventive extract may be prepared in the following manner.

Step 1: Dried Anemarrhena asphodeloides and Aralia elata are pulverized together to prepare powder. To the powder, water or organic solvent, such as alcohol, is added, followed by extraction.

In this step, when a water is used as the extraction solvent, the plant powder will be extracted by heating in a hot bath or at a temperature of more than 120° C. and a pressure of 15 psi. When an alcohol is used as the extraction solvent, the plant powder will be extracted at room temperature. The alcohol used as the extraction solvent is preferably an alcohol having 1 to 6 carbon atoms.

Step 2: The extract obtained in step 1 is centrifuged to remove the precipitate.

Step 3: The filtrate separated in step 2 is extracted with an organic solvent, such as chloroform, hexane, dichloromethane or cyclohexane, and preferably chloroform or hexane thereby removing impurities, such as resin or fibroid material, and the aqueous layer is purified with talc and the like, thus obtaining the desired extract.

The extract is preferably freeze-dried and powdered.

Meanwhile, to confirm the safety of the water or organic solvent extract of Anemarrhena asphodeloides and Aralia elata, the extract was administered to mice, and measured for the acute toxicity of the drug and subjected to histopathological tests (see Test Example 4). As a result, it could be found that the extract is a very safe substance that shows little or no toxicity (see Table 5).

Accordingly, the present inventors formulated the inventive water or organic solvent extract of Anemarrhena asphodeloides and Aralia elata into cream or gel-type preparations, and clinically tested the preparations in order to examine the effects of the extract (see Test Examples 5 and 6). As a result, it could be found that the inventive water or organic solvent extract of Anemarrhena asphodeloides and Aralia has the effect of treating inflammatory skin diseases, such as seborrheic dermatitis, acne, atopic dermatitis and contact dermatitis (see FIG. 1 and Table 6).

Accordingly, the inventive water or organic solvent extract of Anemarrhena asphodeloides and Aralia elata can be used for the prevention or treatment of inflammatory skin diseases. Because the inventive water or organic solvent extract of Anemarrhena asphodeloides and Aralia elata is a very safe substance showing little or no toxicity in vivo, it can be prepared in various forms, including cosmetic compositions, food compositions and pharmaceutical compositions.

As used herein, the term “inflammatory skin diseases” refers to diseases accompanied with a series of clinical signs and symptoms, such as itch, edema, erythema and abrasion are induced by various stimulative factors that cause a series of inflammatory reactions in the skin epithelium. Examples of the inflammatory skin diseases may include, but are not limited to, acute and chronic eczema, contact dermatitis, atopic dermatitis, seborrheic dermatitis, lichen simplex chronicus, intertrigo, dermatitis exfoliativa, papular urticaria, psoriasis, solar dermatitis and acne. Preferred examples of inflammatory skin disease may include contact dermatitis, atopic dermatitis, seborrheic dermatitis and acne.

The content of the water or organic solvent extract of Anemarrhena asphodeloides and Aralia elata comprised in the inventive composition, which is necessary to achieve the desired object, will vary depending on which step-extract is applied to the inventive composition. To obtain a therapeutic effect by the application of the inventive water or organic solvent extract of Anemarrhena asphodeloides and Aralia elata, the extract is preferably used in an amount of 0.001-10.0% by weight based on the weight of the composition. More specifically, if an extract collected just after solvent extraction and filtration is used, it will preferably be contained in an amount of 0.05-10.0% by weight on the basis of a liquid phase. If it is contained in an amount of less than 0.05% by weight, its effect will not be sufficient to achieve the desired object, and if it is contained in an amount of more than 10.0% by weight, it is will be uneconomical because an increase in its effect caused by an increase in its content will not be obtained, and also, it will reduce, the stability of the resulting product. Also, in the case of an extract in which the contents of active ingredients in the extract have been selectively increased by a concentration process using a vacuum concentrator and a freeze dryer, its preferred use content will range from 0.001 to 5.0% by weight, on the basis of dry matter. If the use content is out of this range, the same problems as described above for the extract can occur.

The composition comprising the water or organic solvent extract of Anemarrhena asphodeloides and Aralia elata can be prepared in the form of a cosmetic composition and a food composition.

The cosmetic composition can be easily prepared in any method known in the art, using the water or organic solvent extract of Anemarrhena asphodeloides and Aralia elata together with at least one carrier and additives, which are commonly used in the field of preparing cosmetic compositions.

More specifically, the inventive cosmetic composition can be prepared in the form of basic cosmetic compositions (facial cleansers, such as toilet water, cream, essence, cleansing foam and cleansing water, pack and body oil), color cosmetic compositions (foundation, lipstick, mascara, and make-up base), hair product compositions (shampoo, rinse, hair conditioner and hair gel) and soap etc., which comprise the inventive water or organic solvent extract of Anemarrhena asphodeloides and Aralia elata, as an active ingredient, together with a dermatologically acceptable carrier. Examples of the carriers may include, but are not limited to, a skin softener, a skin permeation enhancer, a colorant, an aromatic, an emulsifier, a thickener, and a solvent. Also, the cosmetic composition may further comprise a perfumery, a pigment, a bactericidal agent, an antioxidant, a preservative and a moisturizer, and also a thickener, inorganic salts and synthetic polymer substances, for the purpose of improving physical properties.

For example, the facial cleanser and soap, which comprise the inventive water or organic solvent extract of Anemarrhena asphodeloides and Aralia elata, can be easily prepared by adding the water or organic solvent extract of Anemarrhena asphodeloides and Aralia elata to the facial cleanser base and soap base. The cream can be prepared by adding the water or organic solvent extract of Anemarrhena asphodeloides and Aralia elata to a general oil-in-water (O/W) cream base. The cleanser, soap and cream may further comprise a perfumery, a chelating agent, a pigment, an antioxidant and a preservative, and also synthetic or natural materials, proteins, minerals and vitamins, for the purpose of improving physical properties.

Also, the inventive cosmetic composition may further contain keratin-removing agents capable of increasing an improvement effect on inflammatory skin diseases, and, including plant-derived proteases, such as papain, bromelain and microorganism-derived proteases. Particularly, to increase a therapeutic effect on atopic dermatitis, contact dermatitis and seborrheic dermatitis, the inventive cosmetic composition may further contain inflammation-inhibitory substances, such as salicylic acid, and a moisturizer, and to increase a therapeutic effect on acne, it may further contain substances, such as salicylic acid or triclosan.

Also, the food composition may be easily prepared in various forms according to any method known in the art, using the water or organic solvent extract of Anemarrhena asphodeloides and Aralia elata, together with at least one carrier or additive, which are generally used in the field of preparing food compositions. The inventive food compositions include in all possible forms, such as functional food, nutritional supplement, health food and food additives.

For example, in case of the health food, the water or organic solvent extract of Anemarrhena asphodeloides and Aralia elata themselves may be prepared into teas, juices and drinks for drinking, or granulated, capsulized and powdered for ingestion. Also, the health food composition may be prepared in the form of a composition by mixing the inventive water or organic solvent extract of Anemarrhena asphodeloides and Aralia elata with active ingredients known to have the effects of preventing and improving inflammatory skin diseases. Furthermore, in order to the water or organic solvent extract of Anemarrhena asphodeloides and Aralia elata to be used in the form of food additives, the inventive extract may be prepared in the form of powder or a concentrate.

Moreover, the pharmaceutical composition for preventing or treating of inflammatory skin diseases, which comprises the inventive water or organic solvent extract of Anemarrhena asphodeloides and Aralia elata, may comprise the water or organic solvent extract of Anemarrhena asphodeloides and Aralia elata alone or may further comprise at least one pharmaceutically acceptable carrier, excipient or diluent. As used herein, the term “pharmaceutically acceptable” refers to a composition is physiologically acceptable, and when administered to human beings it does not cause allergic reactions or similar reactions.

The inventive pharmaceutical composition for preventing or treating inflammatory skin diseases can be administered to mammals by any means. For example, it can be administered orally or parenterally. The parenteral administration methods may include, but are not limited to, transdermal, subcutaneous, intravenous, intramuscular and intra-abdominal routes. Preferably, the inventive pharmaceutical composition for preventing or treating inflammatory skin disease may be administered transdermally. As used herein, the term “administered transdermally” means that the inventive pharmaceutical composition for preventing or treating inflammatory skin diseases is administered to the cells or skin so that active ingredients contained in the composition are absorbed into the skin and this term is include illinition.

The inventive pharmaceutical composition can be formulated into oral preparations or parenteral preparations depending on the above-described administration methods.

In the case of the oral preparations, the inventive composition can be formulated into powders, granules, tablets, pills, sugar-coated tablets, capsules, liquids, gels, syrups, suspensions, etc. by any method known in the art. For example, the oral preparations may be obtained as tablets or sugar-coated tablets by blending the active ingredient with a solid excipient, crushing the blend, adding suitable adjuvants and then processing the mixture into a granular mixture. Examples of suitable excipients may include sugars including lactose, dextrose, sucrose, sorbitol, mannitol, xylitol, erythritol and maltitol; starches including corn starch, wheat starch, rice starch and potato starch; celluloses including cellulose, methyl cellulose, sodium carboxymethylcellulose and hydroxypropylmethyl cellulose; and fillers including zelatin and polyvinylpyrrolidone. Also, the inventive pharmaceutical composition may, if necessary, contain a disintegrant, such as crosslinked polyvinylpyrrolidone, agar, alginic acid or sodium alginate. Furthermore, the pharmaceutical composition for preventing or treating inflammatory skin diseases may further comprise an anticoagulant, a lubricant, a wetting agent, a perfumery, an emulsifier, and a preservative.

In case of the parenteral preparations, they can be formulated in the form of injections, creams, lotions, external ointments, oils, moisturizers, gels, aerosols, and nasal inhalers by any method known in the art. These preparations are described in the following formulary known to all pharmaceutical chemists: Remington's Pharmaceutical Science, 15th Edition, 1975, Mack Publishing Company, Easton, Pa. 18042, Chapter 87: Blaug, Seymour.

Although the content of the extract of Anemarrhena asphodeloides and Aralia elata in the inventive pharmaceutical composition may vary depending on the concentration or non-concentration of the extract as described above, it is preferably 0.001-10% by weight.

The oral dose of the inventive pharmaceutical composition for preventing or treating inflammatory skin diseases is preferably 1000 mg/day-3000 mg/day and more preferably about 1500 mg/day-2500 mg/day, based on a bodyweight of 60 kg. However, the dose of the inventive composition can be suitably selected depending on various factors, such as administration routes, a patient's age, sex, bodyweight and disease severity of patents.

Furthermore, the present invention provides an antibacterial composition against Propionibacterium acnes, which comprises a water or organic solvent extract of Anemarrhena asphodeloides and Aralia elata as an active ingredient. Although the content of the water or organic solvent extract of Anemarrhena asphodeloides and Aralia elata in the antibacterial composition may vary depending on the concentration or non-concentration of the extract as described above, it is preferably 0.001-10% by weight.

In one test example of the present invention, the inventive water or organic solvent extract of Anemarrhena asphodeloides and Aralia elata was measured for antibacterial activity against Propionibacterium acnes (see Test Example 3). As a result, it could be found that the antibacterial activity of the water or organic solvent extract of Anemarrhena asphodeloides and Aralia elata was 6 to 20-fold higher than that of a single extract of each plant (see Table 4).

Also, the inventive antibacterial composition may comprise, in addition to the extract, a pharmaceutically acceptable carrier, excipient or diluent. Preferred examples of the carrier, excipient or diluent are as described above.

In another aspect, the present invention provides a method for preventing or treating inflammatory skin diseases, which comprising administering to a subject in need thereof an effective amount of the water or organic solvent extract of Anemarrhena asphodeloides and Aralia elata.

In still another aspect, the present invention provides a method for inhibiting the growth of Propionibacterium acnes, which comprising administering to a subject in need thereof an effective amount of the antibacterial composition.

As used herein, the term “subjects” may be animals, and preferably mammals. The subjects may also be animal-derived cells, tissues or organs.

In this regard, although the effective amount may vary depending on the concentration or non-concentration of the extract, it may preferably be in a range of 0.001-10.0% by weight.

In still another aspect, the present invention provides a water or organic solvent extract of Anemarrhena asphodeloides and Aralia elata, for use as an active therapeutic ingredient.

In still another aspect, the present invention provides the use of a composition comprising a water or organic solvent extract of Anemarrhena asphodeloides and Aralia elata, for preparing a agent for preventing or treating inflammatory skin diseases.

In yet another aspect, the present invention provides the use of a composition comprising a water or organic solvent extract of Anemarrhena asphodeloides and Aralia elata, for preparing an antibacterial agent against Propionibacterium acnes.

Hereinafter, the present invention will be described in detail by examples. It is to be understood, however, that these examples are given for illustrative purpose only and are not construed to limit the present invention.

### BRIEF DESCRIPTION OF THE DRAWINGS

FIG. 1 is a photograph showing the acne treatment effect of a water or organic solvent extract of Anemarrhena asphodeloides and Aralia elata according to the present invention.

### BEST MODE FOR CARRYING OUT THE INVENTION

Hereinafter, the present invention will be described in detail by examples. It is to be understood, however, that these examples are given for illustrative purpose only and are not construed to limit the present invention.

### Example 1

### Preparation of Extract of Anemarrhena asphodeloides or Extract of Aralia elata

3000 ml of distilled water was added to 300 g of powder prepared by pulverizing dried Anemarrhena asphodeloides with a pulverizer or 300 g of powder prepared by pulverizing dried Aralia elata with a pulverizer respectively. The powder solution was saturation extracted at a temperature of 121° C. and a steam pressure of 15 lb/in² for 1 hour. The extract was isolated and collected and the residue was removed. The extract was centrifuged to remove the precipitate, and the supernatant was filtered and then concentrated to a total volume of 1500 ml. The concentrate was placed in a separatory funnel, and 400 ml of hexane was added thereto to dissolve resin and fibroid material. The organic solvent layer was isolated and removed. The remaining layer was collected and warmed at 70° C., to which 500 g of talc was then added. The mixture was stirred and filtered in vacuum to remove the talc. The filtrate from which the talc has been removed was filtered and centrifuged to collect the supernatant. The supernatant was freeze-dried and powdered, thus preparing an extract of each of Anemarrhena asphodeloides and Aralia elata.

### Example 2

### Preparation of Water Extract of Anemarrhena asphodeloides and Aralia elata

Dried Anemarrhena asphodeloides and dried Aralia elata were mixed at a weight ratio of 3:2 and pulverized with a pulverizer to obtain powder. Then, 300 g of the powder was taken and prepared into a water extract of Anemarrhena asphodeloides and Aralia elata in the same manner as in Example 1. The water extract was freeze-dried and powdered.

### Example 3

### Preparation of Ethanol Extract of Anemarrhena asphodeloides and Aralia elata

Dried Anemarrhena asphodeloides and dried Aralia elata were mixed at a weight ratio of 3:2 and pulverized with a pulverizer to obtain powder. Three thousand ml of ethanol was added to 300 g of the powder, and the powder solution was extracted at room temperature for 2 days. The extract was isolated and collected and the residue was removed. Then, the extract was concentrated, fractionated, filtered, freeze-dried and powdered in the same manner as in Example 1.

### Test Example 1

### Examination for Anti-Inflammatory Effect of Inventive Extract of Anemarrhena asphodeloides and Aralia elata

The anti-inflammatory effect of the inventive powered extracts prepared in Examples 2 and 3 was examined using carrageenin paw edema and the measurement of PEG2 production.

1-1) Examination of Anti-Inflammatory Effect Using Carrageenin Paw-Edema

Male white rats weighing about 200 g each were divided into a control group, a group, administered with the water extract of Anemarrhena asphodeloides, a group administered with the water extract of Aralia elata, a group administered with the water extract of Anemarrhena asphodeloides and Aralia elata, and a group administered with the ethanol extract of Anemarrhena asphodeloides and Aralia elata, in which each group consists of 7 animals. The control group was intraperitoneally (i.p) injected with physiological saline, and the remaining four test groups were intraperitoneally injected with 100 mg/kg of each of the Anemarrhena asphodeloides extract and Aralia elata extract prepared in Example 1, the water extract of Anemarrhena asphodeloides and Aralia elata prepared in Example 2, and the ethanol extract of Anemarrhena asphodeloides and Aralia elata prepared in example 3. Immediately after completion of the administration of the extract, 0.1 ml of a physiological saline solution containing 1% carrageenin was injected into the skin of the paw sole of the male white rats. After one hour, the volume of paw edema up to the ankle joint was calculated by measured with a plethysmometer, and the inhibitory effect of edema (% inhibition) was determined according to the following equation:
Inhibitory effect of edema(% inhibition)=100−(volume of paw edema in test group/volume of paw edema in control group)×100

In the test results, the inventive water extract of Anemarrhena asphodeloides and Aralia elata, and the inventive ethanol extract of Anemarrhena asphodeloides and Aralia elata, showed the inhibitory effect of edema is 82.3% and 79.3%, respectively and the effect is highest among the test group. These values had a statistically significant difference (p<0.001) relative to 3.2% shown for the control group. Also, the inhibitory effect of edema of the water or organic solvent extract of Anemarrhena asphodeloides and Aralia elata was shown to be higher than the sum of the inhibitory effect of edema for the single administration of the Anemarrhena asphodeloides extract or inhibitory effect of Aralia elata extract, indicating that the inventive water or organic solvent extract has a synergistic effect (see Table 1).

TABLE 1
Inhibitory effect of edema of the inventive water or organic
solvent extract of Anemarrhena asphodeloides and Aralia elata

| | | Injec- | |
|---|---|---|---|
| | Dose | tion | |
| Groups | (mg/kg) | means | % Inhibition |
| Control group | 0.9% | i.p. | 3.2 ± 1.0 |
| | saline | | |
| Group administered with | 100 | i.p. | 44.5 ± 11.3** |
| extract of Anemarrhena | | | |
| asphodeloides | | | |
| Group administered with | 100 | i.p. | 31.2 ± 5.6* |
| extract of Aralia elata | | | |
| Group administered with water | 100 | i.p. | 82.3 ± 4.7*** |
| extract of Anemarrhena | | | |
| asphodeloides and Aralia | | | |
| elata | | | |
| Group administered ethanol | 100 | i.p. | 79.3 ± 8.5*** |
| extract of Anemarrhena | | | |
| asphodeloides and Aralia | | | |
| elata | | | |

***p < 0.001,
**p < 0.01,
*p < 0.05 (relative to control group)

1-2) Examination of Anti-Inflammatory Effect by Measurement of PGE2 Production

The effect of the inventive water or organic solvent extract of Anemarrhena asphodeloides and Aralia elata on the production of PGE2 (prostaglandin E2) in mouse macrophages was examined to measure a concentration at which a 50% inhibitory effect is shown. The PGE2 is a substance synthesized by a COX-2 enzyme in macrophages permeated skin when infected with foreign substances or germs, and the degree of inflammation and the secreted amount of PGE2 are closely connected with each other. Namely, as inflammation becomes more severe, the secretion of PGE2 increases.

Meanwhile, when mouse macrophage cell line Raw264.7 (obtained from Korean Cell Line Bank) is cultured in RPMI medium while it is treated with LPS (lipopolysaccharide) for 16 hours, the production of PGE2 will increase. Thus, 1 hour before the macrophage cell line was treated with LPS, the macrophage cell line was treated with each of the water extract of Anemarrhena asphodeloides or water extract of Aralia elata prepared in Example 1, the water extract of Anemarrhena asphodeloides and Aralia elata prepared in Example 2 and the ethanol extract of Anemarrhena asphodeloides and Aralia elata prepared in Example 3, at varying concentrations of 5 mg/ml, 1 mg/ml, 500 μg/ml, 100 μg/ml, 50 μg/ml and 10 μg/ml, and then measured for the amount of PEG2 produced. In this way, the effect of each of the extracts on the PGE2 production of macrophages caused by treatment with LPS was examined. The amount of PGE2 produced was measured using an ELISA kit (Amersham Biosciences) containing an anti-PGE2 antibody. At this time, a positive control group was treated with aspirin, and a negative control group was treated with RPMI1640 medium. The inhibition of production of PGE2, resulting from treatment with each of the samples, was measured, assuming that the difference in PGE production between the group treated with LPS and the group untreated with LPG is 100%. On the basis of the measured value, the IC₅₀ value was calculated which is the concentration necessary to inhibit the production of PGE2 up to 50%. The calculated IC₅₀ value was used as an indication of the inhibition of the COX-2 enzyme activity.

In the test results, the IC₅₀ value of each of the test groups was significantly lower in the group treated with the water or ethanol extract of Anemarrhena asphodeloides and Aralia elata than that in the group treated with the Anemarrhena asphodeloides extract or Aralia elata extract alone (see Table 2).

From the test results, it could be found that the use of the mixed extract of Anemarrhena asphodeloides and Aralia elata more effectively inhibited the production of PGE2 than the use of the single extract of the Anemarrhena asphodeloides or Aralia elata extract, indicating that the inventive extract can effectively inhibit inflammations.

TABLE 2
IC₅₀ value for inhibition of PEG2 production, caused
by treatment with inventive water or organic solvent extract of
Anemarrhena asphodeloides and Aralia elata

| | IC₅₀ |
|---|---|
| Groups | (μg/ml) |
| Group treated with aspirin | 48 |
| Group treated with extract of Anemarrhena | 377 |
| asphodeloides | |
| Group treated with extract of Aralia elata | 450 |
| Group treated with water extract of Anemarrhena | 136 |
| asphodeloides and Aralia elata | |
| Group treated with ethanol extract of Anemarrhena | 153 |
| asphodeloides and Aralia elata | |

### Test Example 2

### Anti-Inflammatory Effect According to Component Ratio of Anemarrhena asphodeloides and Aralia elata

An anti-inflammatory effect according to the component ratio of Anemarrhena asphodeloides and Aralia elata was examined by carrageenin-induced mouse paw edema in the same manner as in Test Example 1-1).

First, dried Anemarrhena asphodeloides powder and dried Aralia elata powder were mixed with each other at weight ratios of 5:1, 3:2, 1:1, 1:6 and 1:10. Then, 100 g of each of the mixtures was taken and prepared into a water extract of Anemarrhena asphodeloides and Aralia elata in the same manner as in Example 1. The anti-inflammatory effect of each of the extracts was measured in the same manner as in Test Example 1-1).

In the test results, the water or organic solvent extract of Anemarrhena asphodeloides and Aralia elata was significantly excellent in anti-inflammatory action as compared; to the control group. Particularly, the extract prepared using Anemarrhena asphodeloides and Aralia elata at a weight ratio of 3:2 most effectively inhibited carrageenin-induced mouse paw edema (see Table 3).

TABLE 3
Inhibitory effect of edema in mice according to component
ratio of Anemarrhena asphodeloides and Aralia elata

| | Dose | Injection | |
|---|---|---|---|
| Groups | (mg/kg) | route | % inhibition |
| Control group | 0.9% | i.p. | 3.6 ± 1.3 |
| | saline | | |
| Anemarrhena | 100 | i.p. | 65.6 ± 10.1 *** |
| asphodeloides:Aralia elata = | | | |
| 5:1 | | | |
| Anemarrhena asphodeloides to | 100 | i.p. | 82.3 ± 4.7 *** |
| Aralia elata = 3:2 | | | |
| Anemarrhena asphodeloides to | 100 | i.p. | 72.8 ± 9.5 *** |
| Aralia elata = 1:1 | | | |
| Anemarrhena asphodeloides to | 100 | i.p. | 59.5 ± 11.5 *** |
| Aralia elata = 1:6 | | | |
| Anemarrhena asphodeloides to | 100 | i.p. | 53.5 ± 8.5 *** |
| Aralia elata = 1:10 | | | |

*** p < 0.001 (relative to control group)

### Test Example 3

### Examination for Antibacterial Effect of Inventive Water or Organic Solvent Extract of Anemarrhena asphodeloides and Aralia elata Against Propionibacterium acnes

To measure the antibacterial effect of the inventive extract against Propionibacterium acnes, 3.7 g/L of BHI (brain heart infusion) broth was inoculated with a culture of Propionibacterium acnes (KCTC 3314) at a concentration of 1% (v/v). Then, the inoculated broth was treated with each of the water extract of Anemarrhena asphodeloides or water extract of Aralia elata prepared in Example 1, the water extract of Anemarrhena asphodeloides and Aralia elata prepared in Example 2 and the ethanol extract of Anemarrhena asphodeloides and Aralia elata prepared in Example 3 at varying concentrations of 0.1%, 1%, 5%, 10% and 20%. A negative control group was treated with physiological saline. Each of the negative control and test groups was anaerobically cultured at 37° C. for 48-72 hours, and then measured for the absorbance at an O.D. 660 nm to examine the minimum inhibitory concentration (MIC) of each extract against Propionibacterium acnes (see Leyden J J et al., J Am Acad Dermatol, 8:41, 1983; Armold H L. et al., Andrew's Diseases of skin, Clinical dermatology, 8th Ed. WB Saunders Co. Philadelphia, 250-258, 1990; CTFA safety testing guideline, The Cosmetics, Toiletry, and Fragrance Association Inc, Washington D.C., 20023, 1991). As positive control groups, erythromycin and triclosan, which are known to have antiacne effect, were used.

In the test results, the MIC values of erythromycin and triclosan used as the positive control group were similar to that reported in existing literature, thus demonstrating the test reliability (Felmingham D. et al. Drugs Exp. Clin. Res. 13(4):195-9, 1987; Nam C. et al. Skin Pharmacol Appl Skin Physiol. 16(2):84-90, 2003). Meanwhile, the MIC values of the inventive water extract and ethanol extract of Anemarrhena asphodeloides and Aralia elata were shown to be 0.0029 ppm and 0.0061 ppm, respectively. These values are much lower than the MIC value of the group treated with the extract of Anemarrhena asphodeloides or extract of Aralia elata alone, indicating that the inhibitory activity of the inventive water or organic solvent of Anemarrhena asphodeloides and Aralia elata against Propionibacterium acnes was about 6 to 20-fold higher than the extract of each plant (see Table 4).

TABLE 4
Minimum inhibitory concentration against Propionibacterium acnes,
according to treatment with inventive water or organic solvent
extract of Anemarrhena asphodeloides and Aralia elata

| | MIC |
|---|---|
| Groups | (ppm, v/v) |
| Group treated with erythromycin | 0.0005 |
| Group treated with and triclosan | 0.001 |
| Group treated with extract of Anemarrhena asphodeloides | 0.01 |
| Group treated with extract of Aralia elata | 0.075 |
| Group treated with water extract of Anemarrhena | 0.0029 |
| asphodeloides and Aralia elata | |
| Group treated with ethanol extract of Anemarrhena | 0.0061 |
| asphodeloides and Aralia elata | |

### Test Example 4

### Test for Toxicity of Inventive Water or Organic Solvent Extract of Anemarrhena asphodeloides and Aralia elata

4-1). Examination for Lethal Dose of Mouse Administered Orally with Water or Organic Solvent Extract of Anemarrhena asphodeloides and Aralia elata.

To confirm the safety of the water or organic solvent extract of Anemarrhena asphodeloides and Aralia elata according to the present invention, LD₅₀ value (an amount which can kill 50% of the experimental animals) of the drug as the standard index for acute toxicity was determined according to the following method. Thirty six normal ICR mice (male, 22±1 g) were divided into 6 groups consisting of Groups A to F, wherein each group consisting comprises 6 mice. Group A was administered orally with 5 g per kg mouse body weight of the ethanol extract of Anemarrhena asphodeloides and Aralia elata prepared in Example 3. Also, the ethanol extract was orally administered to Group B in an amount of 7.5 g per kg mouse body weight, to Group C in an amount of 10 g per kg mouse body weight, to Group D in an amount of 12.5 g per kg mouse body weight and to Group E in an amount of 15 g per kg mouse body weight. Then, the LD₅₀ value of the ethanol extract administered was determined by the Behrens-Krber method (see Drug Experiments, Japan, p 131, 1960).

In the test results, no animal killed by administering the inventive water or organic solvent extract of Anemarrhena asphodeloides and Aralia elata was shown. Also, no animal was killed even in the group to which the inventive extract was administered in a high dose of 15 g per kg of mouse body weight. Thus, it could be seen that the inventive water or organic solvent extract of Anemarrhena asphodeloides and Aralia elata has an LD₅₀ value of more than 15 g/kg and therefore is a very safe material having little or no toxicity (see table 5).

TABLE 5
Oral lethal dose (LD₅₀) of inventive water or organic
solvent extract of Anemarrhena asphodeloides and Aralia elata

| | | Number of animals killed/ | | |
|---|---|---|---|---|
| Test groups | Dose (g/kg) | number of animals tested | *Z | **d |
| A | 5 | 0/6 | — | — |
| B | 7.5 | 0/6 | 0 | 2.5 |
| C | 10 | 0/6 | 0 | 2.5 |
| D | 12.5 | 0/6 | 0 | 2.5 |
| E | 15 | 0/6 | 0 | 2.5 |

*Z: one-half (½) the number of killed animals at two consecutive doses
**d: a difference between two consecutive doses

4-2) Autopsy and Histopathological Test for Mice Administered Orally with Water or Organic Solvent Extract of Anemarrhena asphodeloides and Aralia elata

The autopsy and histopathological test for mice administered orally with water or organic solvent extract of Anemarrhena asphodeloides and Aralia elata in Test Example 4-1 were conducted in the following manner. After completion of the experiment of Example 4-1, all the viable animals were anesthetized with ether and killed by bleeding. Then, the desired organs were extracted and any abnormality of the organs was visually examined. To conduct the histopathological test, all the dissected organs were fixed in 10% neutral formalin solution for 10 days or more, and then dried, embedded into a paraffin embedding system (Fisher, Histomatic Tissue Processor, 166A) and cut into 5 μl m sections using AO Rotary Microtome, followed by staining with hematoxylin and eosin. Then, the condition of the stained sections was observed.

In the test results, any abnormality in the liver tissue and kidneys of the mice, caused by the administration of the inventive extract, was not found even when the inventive extract was administered in a high dose of 15 g per kg of mouse body weight. In addition, abnormalities in the myocardial cells of the heart, gastrointestinal tracts, pancreas, lungs, spleen, adrenal glands, brains, testis, ovary, bone marrow, etc., caused by the drug administration, was not observed.

Therefore, it could be determined that the inventive water or organic solvent extract of Anemarrhena asphodeloides and Aralia elata shows no side effect resulting from acute toxicity in all the organs, even when the inventive extract is administered in a dose of 15 g per kg of body weight as the maximum dose which can be administered to mice, and further, that it is a safe drug which does not induce toxicity causing damages to organs.

### Example 4

### Preparation of Formulations Comprising Inventive Water or Organic Solvent Extract of Anemarrhena asphodeloides and Aralia elata

4-1) Preparation of Facial Cleanser

To 12 g of a facial cleanser base comprising-6 g of glycerin, 2.0 g of monoalkyl phosphate, 0.5 g of sodium hydroxide solution, 1.5 g of myristic acid and a trace amount of perfumery, the water or ethanol extract of Anemarrhena asphodeloides and Aralia elata prepared in Example 3 was added at a concentration of 0.5% (w/w). The mixture was stirred in a homo-mixer and heated at 60° C. for 3 minutes. The heated material was degassed and cooled to 37° C., thus preparing a facial cleanser composition.

4-2) Preparation of Soap

To 99.5% by weight (including water) of a soap base, the powder of water or ethanol extract of Anemarrhena asphodeloides and Aralia elata prepared in Example 3 was added at a concentration of 0.5% (w/w). The blend was well mixed in a mixer. The mixture was placed in a soap making system where it was extruded, cut and stamped, thus preparing a solid soap composition.

4-3) Preparation of Cream

To 40 g of a cream base comprising oily components, aqueous components and a surfactant, such as 1.5 g of stearic acid, 2.2 g of stearylalcohol, 0.5 g of butyl stearate, 0.5 g of propylene glycol, 2.0 g of glycerin monostearate, 0.3 g of potassium hydroxide, etc., the powder of water or ethanol extract of Anemarrhena asphodeloides and Aralia elata prepared in Example 3 was added at a concentration of 0.05% (w/w). The mixture was well emulsified degassed, filtered and cooled to prepare a cream composition. To the composition, a chelating agent, a perfumery and a pigment were added, and the mixture was prepared into an oil-in-water cream containing a small amount of oily components.

4-4) Preparation of Gel

To 25 g of a gel base comprising 3.0 g of 1,3-butyleneglycol, 0.3 g of polyacrylamide, 1.0 g of polyethyleneglycol/polypropyleneglycol (17/6) copolymer and 0.5 g of sodium hydroxide, the powder of water or ethanol extract of Anemarrhena asphodeloides and Aralia elata prepared in Example 3 was added at a concentration of 0.05%. The mixture was strongly stirred in a homo-mixer, degassed and cooled, thus preparing a gel composition.

4-5) Preparation of Emulsion

To 8 g of an emulsion base comprising 0.5 g of sodium ethylenediaminetetraacetate, 1.0 g of DL-pantenol, 1.5 g of betain, 1.5 g of arachidylalcohol/behenylalcohol/arachidylglycoside, 0.5 g of stearic acid, 1.2 g of cyclomethicone, 0.5 g of isostearyl lactate, 0.3 g of triethanolamine and 0.3 g of polyacrylamine, the powder of water or ethanol extract of Anemarrhena asphodeloides and Aralia elata prepared in Example 3 was added at a concentration of 0.05%. The mixture was strongly mixed in a homo-mixer, degassed and cooled, thus preparing an emulsion composition.

4-6) Preparation of Tablet

250 mg of the powder of water or ethanol extract of Anemarrhena asphodeloides and Aralia elata prepared in Example 3, 260 mg of direct compression lactose, 35 mg of Avicel (microcrystalline cellulose), 15 mg of sodium starch glyconate as a disintegration aid, and 80 mg of direct compression L-HPC (low-hydroxypropylcellulose) as a binder were introduced into an U-shaped mixer and then mixed for about 20 minutes. After completion of the mixing, 10 mg of magnesium stearate as a lubricant was added to and mixed with the mixture for about 3 minutes. The mixture was subjected to quantitative analysis and moisture content analysis and then tableted and film-coated, thus preparing a tablet containing 225 mg of the extract.

4-7) Preparation of Syrup

A suitable amount of white sugar was dissolved in a given amount of water, and 80 mg of paraoxymethylbenzoate and 16 mg of paraoxypropylbenzoate as preservatives were added thereto. 4.5 g of the water or ethanol extract of Anemarrhena asphodeloides and Aralia elata prepared in Example 3 was added thereto and completely dissolved with maintaining the temperature at 60° C. The resulting solution was cooled, after which distilled water was added thereto to make a total volume of 150 ml, thus preparing 3% syrup.

4-8) Preparation of Capsule

450 mg of the water or ethanol extract of Anemarrhena asphodeloides and Aralia elata prepared in Example 3 was mixed with 50 mg of lactose. The mixture was filled in a hard gelatin capsule to prepare a capsule.

### Test Example 5

### Acne Treatment Effect of Inventive Water or Organic Solvent Extract of Anemarrhena asphodeloides and Aralia elata in Clinical Trials

In the daytime, the cream prepared in Example 4, which contains the water or ethanol extract of Anemarrhena asphodeloides and Aralia elata, was applied to the affected part of an acne patient (woman, 20 years old) in a suitable amount two times a day, and at night, the gel prepared in Example 4 was applied to the affected part in a suitable amount one time a day. After one week of the application, the observation of a change in the state of the affected part was conducted. Although the gel preparation has an advantage in that it forms a film upon application to the skin, leading to long-lasting effects, the use of the gel preparation in the daytime is not preferable in terms of appearance and convenience. For this reason, in the daytime, the cream preparation was used.

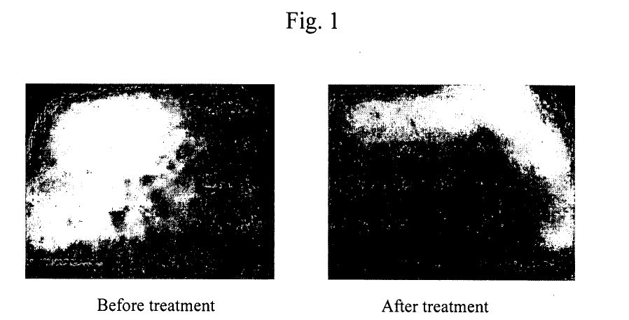

In the test results, it could be observed that, when the cream containing the inventive extract was applied to the affected part of the acne patient, the acne was remarkably improved (see FIG. 1). In other words, the affected part applied with the inventive cream or gel showed a reduction in fat secretion and reductions in the size of acne scars and inflammatory symptoms.

### Test Example 6

### Clinical Treatment Effects of Inventive Water or Organic Solvent Extract of Anemarrhena asphodeloides and Aralia elata on Inflammatory Skin Diseases

The clinical treatment effects of the inventive water or organic solvent extract of Anemarrhena asphodeloides and Aralia elata on inflammatory skin diseases, including seborrheic dermatitis, acne, atopic dermatitis and contact dermatitis, were examined.

For this purpose, to the affected parts of 115 patients (47 men and 68 women, 3 months to 60 years old) having seborrheic dermatitis, acne, atopic dermatitis and contact dermatitis, the cream or gel prepared in Example 4, which contains the inventive water or organic solvent extract of Anemarrhena asphodeloides and Aralia elata, was applied in the same manner as in Test Example 5 (i.e., application of the cream in the daytime and application of the gel at night) 2-3 times a day for 14 days. Then, the treatment effects of the gel or cream were measured. The treatment effects were divided, according to the improved conditions of the patients, into “aggravation”, “no change”, “slightly effective”, “moderately effective” and “significantly effective”.

In the test results, it was shown that the inventive water or organic solvent extract of Anemarrhena asphodeloides and Aralia elata was effective in all the patients having seborrheic dermatitis, acne, atopic dermatitis and contact dermatitis, except for one patient having atopic dermatitis. Also, the inventive water or organic solvent extract of Anemarrhena asphodeloides and Aralia elata was moderately effective or significantly effective in 86% of the patients having various inflammatory skin diseases (see Table 6).

TABLE 6
Treatment effects of inventive water or organic
solvent extract of Anemarrhena asphodeloides and Aralia elata
on inflammatory skin diseases

| Kind of | Patient's response | | | | | |
|---|---|---|---|---|---|---|
| dermatitis | XX | X | Δ | ◯ | ◯◯ | Total |
| Seborrheic | | | 6 | 13 | 10 | 29 |
| dermatitis | | | (21) | (45) | (34) | |
| Acne | | | 4 | 16 | 12 | 32 |
| | | | (13) | (50) | (37) | |
| Atopic | | 1 | 2 | 17 | 11 | 31 |
| dermatitis | | (3) | (6) | (55) | (35) | |
| Contact | | | 3 | 13 | 7 | 23 |
| dermatitis | | | (13) | (56) | (30) | |
| Total | | | | | | 115 |

Unit: persons (%)
XX: aggravation
X: no change
Δ: slightly effective
◯: moderately effective
◯◯: significantly effective

### INDUSTRIAL APPLICABILITY

As can be seen from the foregoing, the inventive composition comprising the water or organic solvent extract of Anemarrhena asphodeloides and Aralia elata shows excellent anti-inflammatory and antibacterial activities and has the effect of preventing or treating various inflammatory skin diseases, including seborrheic dermatitis, acne, atopic dermatitis and contact dermatitis.

1-13. (canceled)

14: An anti-inflammatory cosmetic composition comprising a water or organic solvent extract of Anemarrhena asphodeloides and Aralia elata.

15: An anti-inflammatory food composition comprising a water or organic solvent extract of Anemarrhena asphodeloides and Aralia elata.

16: The composition of claim 14, wherein the organic solvent is an alcohol having 1 to 6 carbon atoms.

17: The composition of claim 14, wherein the component ratio of Anemarrhena asphodeloides and Aralia elata is 1-10:1-15.

18: The composition of claim 14, wherein the water or organic solvent extract of Anemarrhena asphodeloides and Aralia elata is comprised in an amount of 0.001-10.0% by weight.

19: A pharmaceutical composition for preventing or treating inflammatory skin diseases, which comprises a water or organic solvent extract of Anemarrhena asphodeloides and Aralia elata.

20: An antibacterial composition against Propionibacterium acnes, which comprises a water or organic solvent extract of Anemarrhena asphodeloides and Aralia elat.

21: The composition of claim 19, wherein the inflammatory skin diseases are selected from the group consisting of acute and chronic eczema, contact dermatitis, atopic dermatitis, seborrheic dermatitis, lichen simplex chronicus, intertrigo, dermatitis exfoliativa, papular urticaria, psoriasis, solar dermatitis, and acne.

22: A method for preventing or treating inflammatory skin diseases, which comprises administering to a subject in need thereof an effective amount of a water or organic solvent extract of Anemarrhena asphodeloides and Aralia elat.

23: A method for inhibiting the growth of Propionibacterium acnes, which comprises administering to a subject in need thereof an effective amount of a water or organic solvent extract of Anemarrhena asphodeloides and Aralia elat.

24: A method for preparing an agent for preventing or treating inflammatory skin diseases comprising using a water or organic solvent extract of Anemarrhena asphodeloides and Aralia elat.

25: A method for preparing an antibacterial agent against Propionibacterium acnes comprising using a water or organic solvent extract of Anemarrhena asphodeloides and Aralia elat.
